(19) United States
(12) Patent Application Publication
(10) Pub. No.: US 2014/0303573 A1
(43) Pub. Date: Oct. 9, 2014

(54) DIAPERS, DIAPER WIPES AND DIAPER PADS WITH ACTIVE INGREDIENTS INTEGRATED THEREIN

(71) Applicants: Timothy Matthew LaCroix, Morristown, NJ (US); Margaret Anne LaCroix, Morristown, NJ (US)

(72) Inventors: Timothy Matthew LaCroix, Morristown, NJ (US); Margaret Anne LaCroix, Morristown, NJ (US)

(21) Appl. No.: 14/214,306

(22) Filed: Mar. 14, 2014

Related U.S. Application Data
(60) Provisional application No. 61/802,050, filed on Mar. 15, 2013.

(51) Int. Cl. A61F 13/84

(52) U.S. Cl. CPC A61F 13/8405; A61F 2013/15081
USPC 604/307; 604/308

(57) ABSTRACT

A diaper, diaper sheet insert and/or a diaper wipe sheet with active ingredients integrated therein, including ointments for treating diaper rash, baby oils and lotions for treating dry skin, and/or powder treatments for absorbing moisture, including baby powder, corn starch powders and the like. In a first embodiment, the invention provides a diaper having active ingredients integrated therein comprising a skin-contact surface, at least one inner absorption layer, an outer most layer, and a barrier wall having an active ingredients segment and preventing contact of the active ingredients with the diaper's skin-contact surface. The active ingredient may be applied as a coating or integrated as fine particles within the barrier wall. As diaper is worn, or the active ingredient wipe sheet is wiped on the skin, the skin-contact surface comes into contact with skin, transferring active ingredients onto the skin.

This is a nonprovisional patent application of provisional application Ser. No. 61/802,050, filed Mar. 15, 2013 for “Diapers, Diaper Wipes And Diaper Pads With Active Ingredients Integrated Therein”, the disclosure of which is hereby incorporated in its entirety by reference thereto.

### BACKGROUND OF THE INVENTION

1. Field of the Invention

The present invention relates to a diaper, a wipe sheet and/or a diaper sheet insert having active ingredient integrated therein that is appointed to either be wiped on the body and/or inserted into a diaper.

2. Description of the Prior Art

Diaper rash affects virtually all babies at one time or another. The rash ranges from mild outbreaks with few red bumps, while still uncomfortable for the infant, or can be sever with bleeding sores, causing severe pain and discomfort for the infant and worry to the parent or caretakers. Particular problems can arise during overnight wearing of the diaper, as the infant urinates frequently, saturating the diaper with uric acid, and changing of the diaper generally occurs in the morning when the infant/toddler awakens. Even still, where a diaper soiled by an infant's bowl movement is not immediately changed, due to circumstances such as diarrhea, an overnight soiling, traveling or the parent is unaware of the soiling, the infant/toddler's bottom is extremely vulnerable to a diaper rash outbreak.

Despite the problems associated with diaper rash and general maintenance of an infant's bottom, the only recourse is to topically treat the infant's bottom with lathering of the lotion, ointment, and oil or baby powder on the bottom while diapering the infant. Powders and creams or lotions are frequently applied to an infant's bottom to ensure that the infant's skin remains healthy. A plethora of compositions for topical treatments have been provided wherein lotion, ointment or baby powder are applied to the bottom of the infant while diapering, before securing the diaper.

However, such topical application during diapering requires the caregiver/parent to have to fumble with ointment containers/tubes when diapering an infant, which can be quite a challenge when the infant is very active and not wanting to lay still on a changing table or surface. During travel, such ointments are just one more item that needs to be toted around in the diaper bag and dealing with same when using a restroom changing table can be quite difficult. In fact, the situation can be dangerous, as the caregiver must take both hands off of the infant to open the ointment container, leading to the risk of the infant rolling from the changing surface. Moreover, the topical application results in residual ointment or lotion being deposited on the caregiver's fingers. In a perfect situation the caregiver would be able to immediately wash his/her hands. But in reality, the caregiver does not always have that luxury, as the infant cannot be left on the changing table alone while the caregiver goes to wash his/her hands, and it is very difficult washing one's hands while holding an infant. During travel, a sink may not even be readily available, as parents often must change the infant's/baby's diaper in a vehicle or stroller. Realistically, the ointment cannot be immediately washed from the hands after every application, and is transferred onto clothing, or worse, onto bottles and nipples being prepared for feeding the infant, or onto the breast, ultimately risking ingesting by the infant.

Integration of substances within diapers has been found to interfere with absorption capabilities owing to the absorption of the substance into the absorptive layers of the diaper. Interference with absorptive properties currently renders such diapers as subpar in comparison with their non-substance integrated counter parts. As the substance becomes meshed within the absorptive surface of the diaper, the ability to absorb urine and fecal matters is decreased. Another problem associated with diapers with substances with substances, such as lotions, associated therewith is the containment and subsequent displacement of the substance during packaging and transport until ultimate use by the end-user.

Notwithstanding the efforts of prior art workers, there remains a need in the art for a diaper wherein an oil, powder and or ointment is integrated therein in a manner so that the substance does not disperse during shipping and storage, and that does not interfere with absorption, so that direct application is not necessary and the caregiver or parent merely attaches the diaper on the infant and direct delivery of the ointment, oil or powder to the infant's bottom results as the diaper is worn. In addition, there exists an art recognized need for a diaper insertion pad that includes an ointment, oil or powder integrated therein that can readily be placed in a diaper to provide protection to the infant's bottom and deliver active ingredients thereto. Further, there is a need in the art for a baby wipe wherein an active ingredients or lotion is integrated therein, so that the wiper can directly apply active ingredients to an infant's bottom as it is being utilized to wipe the infant's bottom. These applications would fill a need in the art by providing protection to the infant's bottom as the diaper is worn, while further providing the parent or caretaker with the ability to diaper an infant with minimum effort and with minimum items to carry.

### SUMMARY OF THE INVENTION

The present invention provides a diaper, diaper pad insert and/or a diaper wipe with active ingredients integrated therein, including ointments for treating diaper rash, baby oils and lotions for treating dry skin, and/or powder treatments for absorbing moisture, including baby powder, corn starch powders and the like. Specifically, the present invention provides a diaper, diaper pad insert and/or diaper wipe in a construct wherein the active ingredients do not interfere with absorptive properties of the diaper, etc. Further, the present invention provides a diaper, diaper pad insert and/or diaper wipe structure that prevents leakage or disbursement of the ointment onto other areas of the diaper/diapers therewith packaged to keep containment of the ointment. More particularly the subject invention provides a diaper and/or diaper wipe sheet and/or diaper sheet insert with active ingredients integrated therein including diaper rash ointment for treating diaper rash, baby oils and lotions for treating dry skin, and/or powder treatments for absorbing moisture, including baby powder, corn starch powders and the like Direct application of the diaper rash ointment, baby oil, lotion and/or powder is achieved while the diaper is being donned and worn as the active ingredient segment is wiped against the infant's body the active ingredients are transferred onto the infant's skin.

In the embodiments concerning the diaper and diaper sheet insert, during wear, the diaper (and/inserted diaper pad) with active ingredients integrated therein comes into contact with the infant's bottom and transfers the active ingredients thereto. Through long intervals, the infant is protected and the risk of developing (or worsening) diaper rash conditions is mitigated. The diaper should optimally be changed as soon as possible after each soiling as a usual diaper, but the diaper (and/inserted diaper pad) herein facilitates in protecting the infant's body during wear and provides advantages during the diapering process.

Advantageous include: (i) protecting an infant from diaper rash during prolonged periods where the infant is not changed—such as during the night, extended periods of sleep, naptime, or during travel; (ii) convenience during the diapering process, allowing the caregiver to simply place the diaper on the child without the need to apply separate ointments, lotions, and/or powder, which can be very advantageous when dealing with a squirming toddler; (iii) provides the caregiver the ability to apply the ointment, or other medicament, without using their hands so that their hands do not have residual ointment or lotion on them; and (iv) results in a lighter, more compact diaper bag, as less items are necessary to carry along in the bag, so that the caregiver will not have to tote around lotions, ointments and powders in the bag, instead the caregiver will need to only carry the diaper and/or diaper wipes with active ingredients integrated therein.

The caregiver could carry different active ingredient diapers (and/or diaper pads), for example, carrying two or three with ointment, and two or three with powder within a single diaper bag. The invention herein is not appointed to replace the frequency of diaper changing, for the diaper should optimally be changed as soon as possible after being soiled to best prevent diaper rash or irritation. However, the diaper herein will provide the ability to diaper an infant in a fast convenient manner, wherein the caregiver simply places the diaper on the infant, without the step of separately applying ointment. This can be very advantageous when dealing with a squirming infant or toddler. Moreover, the caregiver does not have to deal with diaper rash ointment or the like on the hands, which can be difficult to immediately wash if in a location lacking a sink or if dealing with an infant on a changing table, which cannot be left to attend to hand washing. Further, if the diaper is not immediately changed, the ointment transfer from the diaper will facilitate in preventing the rash and mitigate the severity of a potential rash.

In a first embodiment, the invention provides a diaper having a active ingredients integrated therein comprising a skin-contact surface appointed for contact with an infant's bottom, at least one inner absorption layer, an outer most layer, and a barrier wall located on top of the skin-contact surface, wherein the barrier wall has an active ingredients segment thereon. The active ingredient segment includes active ingredients associated therewith and is adapted for contact and direct delivery to the infant's bottom when the diaper is placed on and worn by the infant. The active ingredient segment is located on top of the barrier wall.

The barrier wall is preferably a very thin impervious layer preventing contact of the active ingredient segment directly on the top surface of the diaper. In this manner, the active ingredient is not directly located on the top diaper surface and thus absorption interference issues of the active ingredient ointment are mitigated. Specifically, preferably the barrier wall is a thin layer that is substantially dissolvable when in contact with urine and/or body fluids and/or body heat so that as the active ingredients are gradually rubbed on to the infants bottom while initially wearing the diaper, the barrier wall or barrier membrane dissolves so that it does not itself interfere with diaper absorbency. The barrier wall may be a thin layer comprised of a starch, and/or a gelatin.

Active ingredients are coated on the barrier wall so that the active ingredients come into contact with the infant's skin when the diaper is being worn or when it is being placed on. As the infant wears the diaper and moves around, the skin-contact surface comes into contact with the infant's body, transferring active ingredients associated with the active ingredient segment onto the infant's body. It should be noted that although the word “infant” is utilized herein, the subject invention's diapers, diaper wipe sheets, and/or diaper sheet inserts can be constructed of varying sizes so that the devices can be used for young children, adults, elderly, or those in need of discrete protection. This active ingredient may be a lotion, baby oil or petroleum jelly for treatment or prevention of dry skin. The active ingredients may be a hemorrhoid cream, vaginal treatment cream, medicated cream for personal hygienic issues, or a jock itch cream/ointment. Most preferably, the active ingredient is a diaper rash ointment for treatment and prevention of diaper rash. A powder treatment for absorption of moisture may be provide as the active ingredient, and may include baby powders, particularly corn starch based powders. The active ingredient segment may be integrated within the portion of the skin-contact surface so that it is interstitially integrated within fibers of the skin-contact surface to provide transfer of the active ingredients. Alternatively, the active ingredient segment is applied as a coating upon the portion of the skin-contact surface to provide transfer of the active ingredients, the coating being comprised of at least one active ingredient layer. The portion of the skin-contact surface with the active ingredient segment integrated therein could be the entire surface thereof, or may simply be a zone or section of the skin-contact surface, such as a soiling zone extending between the crotch area and the buttocks area of an infant being diapered.

In another embodiment, a diaper sheet insert having active ingredients integrated therein is provided. The diaper sheet is a thin sheet and is separate from a diaper and is adapted to be secured within a diaper when the diaper is appointed to be used on an infant. Thus, the diaper sheet inserts can be sold in conjunction with a particular diaper package, or can be sold separate from the diaper package. In this manner, one can receive some of the benefits of a diaper with active ingredients integrated therein, as disclosed herein in the aforementioned embodiment, by simply inserting the diaper sheet insert within a conventional diaper. The diaper sheet insert comprises an ultra-thin sheet having top wall appointed for contact with an infant's bottom and a bottom wall having an adhesive coating applied thereto that is appointed to be attached to a skin-contact surface of a diaper. An active ingredient segment is provided that is located in association with the top layer of the sheet. The active ingredient segment includes active ingredients integrated therein and is adapted for contact and direct delivery to the infant's skin when the diaper pad is secured into the diaper, and the diaper is placed on and worn by the infant. The active ingredients may be selected from a group consisting of baby oil, petroleum jelly or lotion for treatment or prevention of dry skin; hemorrhoid treatment; vaginal treatments; jock itch treatments; or the like. Preferably, the active ingredient is a diaper rash ointment for treatment or prevention of diaper rash. In another embodiment, the active ingredient is a powder treatment for absorption of moisture and the active ingredient segment comprises an inner powder releasing pouch in association with a top layer of the pad.

Another embodiment provides a diaper wipe sheet having active ingredients integrated therein. The diaper wipe sheet comprises a top wipe layer appointed for contact with an infant's skin, a bottom layer, and an active ingredient segment located in association with at least one portion of the top wipe sheet layer. The active ingredient segment includes active ingredients therein and is adapted for contact and direct delivery of the active ingredients to the infant's bottom when the wipe is wiped against the infant.

### BRIEF DESCRIPTION OF THE DRAWINGS

The invention will be more fully understood and further advantages will become apparent when reference is had to the following detailed description of the preferred embodiments of the invention and the accompanying drawings, in which:

FIG. 1a illustrates a top view of an embodiment of the diaper having active ingredients integrated therein comprising a skin-contact surface appointed for contact with an infant's bottom, at least one inner absorption layer, an outer most layer, and an active ingredient segment, wherein the portion of the skin-contact surface appointed with the active ingredient segment is a soiling zone extending from the front area of the diaper past the buttocks area;

FIG. 1b illustrates an embodiment, via a side cross-sectional view of the diaper having active integrated therein of FIG. 1a taken along line x-x, wherein the active ingredient segment is integrated within the portion of the skin-contact surface;

FIG. 1c illustrates an embodiment, via a side cross-sectional view of the diaper having active integrated therein of FIG. 1a taken along line x-x, wherein the active ingredient segment is an active ingredient coating applied on the top of the portion of the skin-contact surface;

FIG. 2a illustrates a top view of an embodiment of the diaper having active ingredients integrated therein comprising a skin-contact surface appointed for contact with an infant's bottom, at least one inner absorption layer, an outer most layer, and an active ingredient segment, wherein the active ingredient segment is an active ingredient coating applied on the top of the portion of the skin-contact surface (as shown in FIG. 1c) and a removable peel tab is located thereon;

FIG. 2b illustrates an embodiment, via a side cross-sectional view of the diaper having active integrated therein of FIG. 2a taken along line y-y, showing the active ingredient segment coating applied on the top of the portion of the skin-contact surface and the removable peel tab in place, appointed to be removed when the diaper is being donned;

FIG. 3 illustrates a view of an embodiment wherein the barrier wall, and visa vie the active ingredient segment, is a raised surface located on said skin-contact surface of said diaper and is shaped as a decorative design;

FIG. 4a illustrates a top view of an embodiment of the diaper having active ingredients integrated therein wherein the barrier wall consists essentially of a plurality of separate barrier wall segments that are closely dispersed but separate in relation to one another;

FIG. 4b illustrates a cross-sectional view taken along line z-z;

FIG. 4c illustrates a cross-sectional view taken along line w-w;

FIG. 5 illustrates a top view of an embodiment of the diaper having active ingredients integrated therein wherein the barrier wall is formed as an accordion shaped portion having channels and peaks formed therein;

FIG. 6 illustrates a top view of an embodiment of the diaper having active ingredients integrated therein wherein the barrier wall is inset within the skin-contact surface of the diaper at a position below the skin-contact surface.

FIG. 7a illustrates a top view of an embodiment of the diaper active ingredient insert having active ingredients integrated therein adapted to be adhered within a diaper as needed by a care giver;

FIG. 7b illustrates the diaper active ingredient insert adhered onto a diaper.

### DETAILED DESCRIPTION OF THE INVENTION

The present invention provides a diaper, diaper sheet insert and/or a diaper sheet wipe with active ingredients integrated therein, including ointments for treating diaper rash, baby oils and lotions for treating dry skin, and/or powder treatments for absorbing moisture, including baby powder, corn starch powders and the like. Direct application of the diaper rash ointment, baby oil, lotion and/or powder is achieved while the diaper is being donned and worn as the active ingredient segment is wiped against the infant's body the active ingredients are transferred onto the infant's skin.

Topical application during diapering requires the caregiver/parent to have to fumble with ointment containers/tubes when diapering an infant, which can be quite a challenge when the infant is very active and not wanting to lay still on a changing table or surface. During travel, such ointments are just one more item that needs to be toted around in the diaper bag and dealing with same when using a restroom changing table can be quite difficult. In fact, the situation can be dangerous, as the caregiver must take both hands off of the infant to open the ointment container, leading to the risk of the infant rolling from the changing surface. Moreover, the topical application results in residual ointment or lotion being deposited on the caregiver's fingers. In a perfect situation the caregiver would be able to immediately wash his/her hands. But in reality, the caregiver does not always have that luxury, as the infant cannot be left on the changing table alone while the caregiver goes to wash his/her hands, and it is very difficult washing one's hands while holding an infant. During travel, a sink may not even be readily available, as parents often must change the infant's/baby's diaper in a vehicle or stroller. Realistically, the ointment cannot be immediately washed from the hands after every application, and is transferred onto clothing, or worse, onto bottles and nipples being prepared for feeding the infant, or onto the breast, ultimately risking ingesting by the infant.

Integration of substances within diapers has been found to interfere with absorption capabilities owing to the absorption of the substance into the absorptive layers of the diaper. Interference with absorptive properties currently renders such diapers as subpar in comparison with their non-substance integrated counter parts. As the substance becomes meshed within the absorptive surface of the diaper, the ability to absorb urine and fecal matters is decreased. Another problem associated with diapers with substances with substances, such as lotions, associated therewith is the containment and subsequent displacement of the substance during packaging and transport until ultimate use by the end-user.

The diaper herein provides a barrier wall having an active ingredient segment thereon that is positioned within the diaper and is structured to prevent the active ingredients from leaking through and becoming absorbed within the diaper itself. Absorption of the active ingredient itself within the diaper's skin contact surface disadvantageously interferes with the diapers ability to absorb urine/fecal matter. Owing to the construction of the diaper herein, a barrier wall is provided that prevents absorption of the active ingredients into the interstices of the diaper's skin contact surface and visa vie absorptive layer. In a first embodiment, the barrier wall herein is provided by way of a thin impervious substantially non-porous surface that is placed on a very small area of the diaper forming the active ingredient segment's protective zone. This zone is small in area as it is provided to merely address the genital area and small segment of the buttocks area of the diaper and thus the zone is not provided in a large area of the diaper. The barrier wall is provided as thin layer and the active ingredient segment via active ingredients are then sprayed or applied thereto. A protective external covering is then provided over the active ingredient segment to prevent the active ingredients from rubbing onto other parts of the diaper during packaging and transport.

In this embodiment, the barrier wall may be simply rolled on or sprayed onto a small area of the diaper. Next, the active ingredients are then rolled on or sprayed onto the barrier wall, followed by a protective exterior layer to prevent the active ingredients from rubbing off unintentionally during packaging and storage of the diaper. Preferably, the active ingredient segment is a tacky surface formed as a coating that is to be directly and immediately wiped onto an infant when the diaper is being placed on. The barrier wall may be a thin polymer or silicone film. Alternatively, the barrier wall may be composed of a material that dissolves in urine and/or bodily fluids. The barrier wall may be composed of a thin starch layer; and/or a thin gelatin layer. Owing to the dissolvability of the barrier wall, a very small area wherein the barrier is applied, and the transfer of the ointment containing the active ingredients to the infant's skin when diapering, neither the barrier wall nor residual ointment embeds to the skin-contact surface of the diaper and therefore, there is minimal interference with absorptive properties of the diaper.

In another embodiment the barrier wall is formed as an upraised layer. Preferably, the upraised layer is formed as an accordion portion having peaks and channels. Diaper rash ointment is located on the peaks, while the channels do not contain ointment. Even more preferably, the channels are composed of an absorptive material. As the diaper is placed on the infant, the ointment on the peaks is wiped against the infant's bottom. When the infant urinates, urine flows to the absorptive channels and some urine is immediately thereby absorbed by the channels. Other urine bypasses the channels and is absorbed in the diaper. In this manner, not only is the diaper's absorptive properties retained, but the diaper's absorptive ability is actually enhanced owing to the additional absorptive ability of the barrier wall's channels. In a similar embodiment, the barrier wall is formed as an upraised layer formed as a raised design or pattern. Diaper rash ointment is coated on the raised design or pattern.

The diaper sheet insert is provided to be inserted within an existing diaper. The sheet insert is a thin sheet that has a top wall with a coating of ointment thereon, and a bottom wall with an adhesive thereon. The adhesive layer is simply adhered to the diaper and the ointment layer is thus exposed to the infant's skin. The diaper sheet is a small sheet—preferably one to two inches in diameter—so that it only takes up a very small area of the diaper and is appointed to be placed in the crotch/buttox area. More than one sheet can be placed in the diaper if the infant is displaying a large area of diaper rash.

The diaper sheet wipe is composed of a thin sheet that is simply opened, and wiped on an infant when diaper rash ointment is needed. The thin sheet is simply discarded after wiping it against the infant.

In the embodiments concerning the diaper and diaper sheet, during wear, the diaper (and/inserted diaper pad) with active ingredients integrated therein comes into contact with the infant's bottom and transfers the active ingredients thereto. Through long intervals, the infant is protected and the risk of developing (or worsening) diaper rash conditions is mitigated. The diaper should optimally be changed as soon as possible after each soiling as a usual diaper, but the diaper (and/inserted diaper pad) herein facilitates in protecting the infant's body during wear and provides advantages during the diapering process. In the embodiments concerning the diaper wipe, the user simply utilizes the wipes to clean the infant, and when doing so the active ingredients are transferred to the infant's bottom. Preferably, the caregiver or parent utilizes a conventional wipe to clean the soiled infant, and then utilizes the diaper wipe with active ingredients thereon of the subject invention on the infant to distribute the active ingredients thereon right before the diaper is placed onto the infant, as the last, final wipe.

Advantageous include: (i) protecting an infant from diaper rash during prolonged periods where the infant is not changed—such as during the night, extended periods of sleep, naptime, or during travel; (ii) convenience during the diapering process, allowing the caregiver to simply place the diaper on the child without the need to apply separate ointments, lotions, and/or powder, which can be very advantageous when dealing with a squirming toddler; (iii) provides the caregiver the ability to apply the ointment, or other medicament, without using their hands so that their hands do not have residual ointment or lotion on them; and (iv) results in a lighter, more compact diaper bag, as less items are necessary to carry along in the bag, so that the caregiver will not have to tote around lotions, ointments and powders in the bag, instead the caregiver will need to only carry the diaper and/or diaper wipes with active ingredients integrated therein.

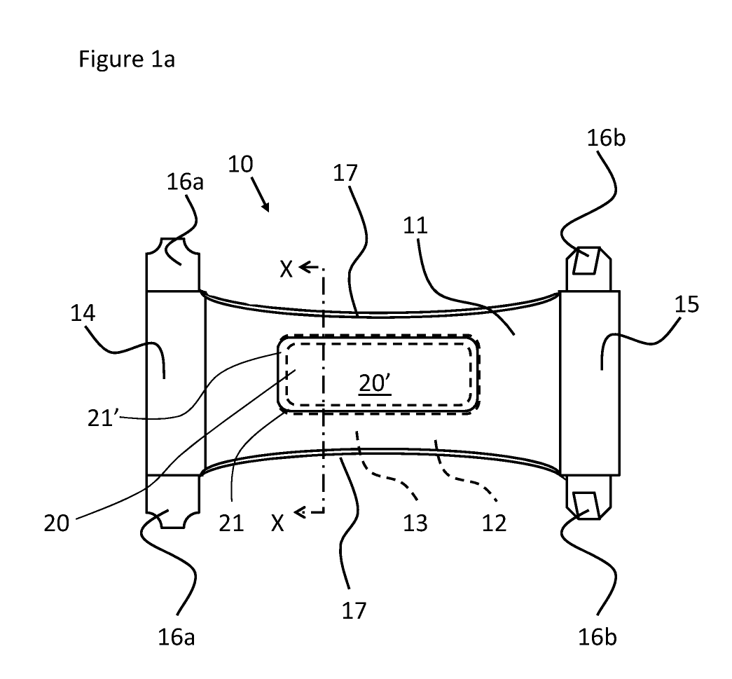

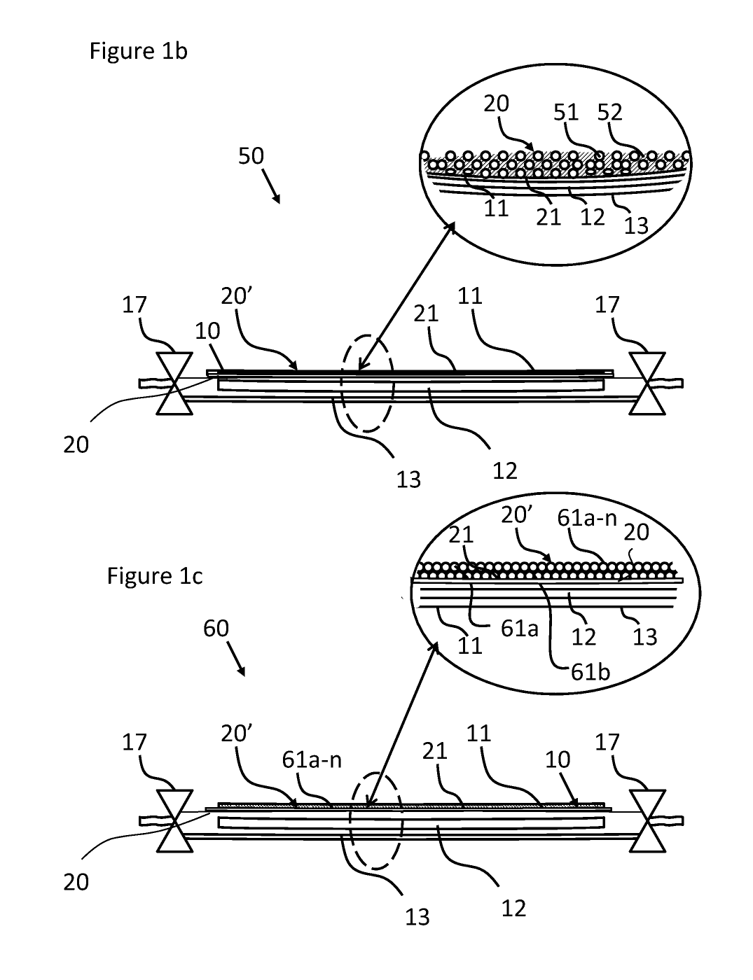

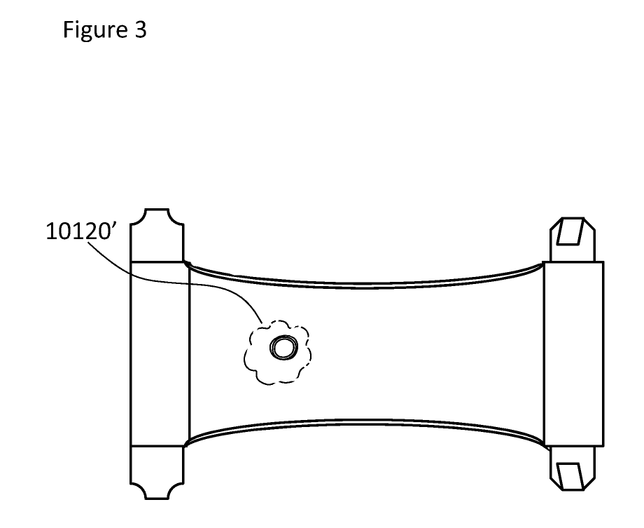

FIG. 1a illustrates a top view of an embodiment of the diaper having active ingredients integrated therein, shown generally at 10. Diaper 10 comprises a skin-contact surface 11 appointed for contact with an infant's bottom, at least one inner absorption layer 12, and an outer most layer 13. Diaper 10 is constructed having a front waist band 14 and a back waist band 15 with mating attachment means 16a, 16b, and leg perimeter zones 17. Diaper 10 further comprises a barrier wall 20 having an active ingredient segment 20′ located over only a portion 21 of skin-contact surface 11, herein portion 21 is shown in a soiling zone extending from an area near the front waist band 14 past the crotch area and into the buttocks area towards the back waist band 15. The portion 21 with the active ingredient segment 20′ thereon/therewith preferably only covers a very small area of the skin contact layer 11. Preferably, portion 21 with active ingredient segment 20′ thereon/therewith is a soiling zone extending from a crotch area to a buttocks area of the diaper so that the active ingredient segment provides contact and dissemination of the active ingredients to crotch and buttocks areas of the infant while the diaper is being donned and worn, as is generally shown in this figure. In another embodiment, the portion 21 may simply be provided as a buttocks zone extending over a buttocks area of the diaper so that the active ingredient segment provides contact and dissemination of the active ingredients to the buttocks of the infant while the diaper is being donned and worn. (See FIGS. 3a and 3b). The active ingredient segment 20′ preferably ranges from about 0.5′ to about 3″ in width; and from about 0.5″ to about 3″ in length. Preferably, the active ingredient segment 20′ has an active ingredient depth or thickness, preferably ranging from 0.05″ to about 0.5″ in depth or thickness. In this manner, the active ingredient segment 20′ is provided as a dab that is essentially meant to be wiped directly onto the infant's bottom during diapering without the need to use the caregiver's hand directly in the active ingredient or directly on the infant's bottom.

The active ingredient segment 20′ includes active ingredients associated therewith and is adapted for contact and direct delivery to the infant's bottom when the diaper 10 is placed on and worn by the infant. The active ingredient segment 20′ is located in association with the barrier wall in turn located on skin-contact surface 11 of the diaper 10 so that the active ingredients coated upon or integrated within the skin-contact surface 11 via active ingredient segment 20′ comes into contact with the infant's bottom. As the infant wears the diaper and moves around, the skin-contact surface 11, and visa vie the active ingredient segment 20′, comes into contact with the infant's body, transferring active ingredients associated with the active ingredient segment 20′ onto the infant's body. This active ingredient may be a lotion, baby oil or petroleum jelly for treatment or prevention of dry skin. Most preferably, the active ingredient is a diaper rash ointment for treatment and prevention of diaper rash. The active ingredient is preferably a coating of a petrolatum or jelly like substance that is directly ready to be wiped on the infant's bottom at diapering for immediate treatment and protection. A powder treatment for absorption of moisture may be provide as the active ingredient, and may include baby powders, particularly corn starch based powders. A protective exterior covering 21′ is provided over the active ingredient segment 20′ to completely encapsulate the active ingredient segment 20′ on the barrier wall 20 so that the active ingredient segment 20′ cannot seep onto other parts of the diaper or other diapers during packaging and transport.

FIGS. 1b and 1c illustrated embodiments of the application of the active ingredient segment 20′ as integrated with skin contact layer 11 of the diaper 10 shown in FIG. 1a as taken along line x-x. Particularly, FIG. 1b illustrates an embodiment wherein the active ingredient segment 20 is integrated within the portion 21 of the skin-contact surface 11, shown generally at 50. In this manner, the integration may be achieved during the manufacturing process by various methods, including by spray, or micro-spray, application onto the skin contact layer 11 during production of the diaper 10. Particles of the active ingredient 51 thus result so that active ingredient segment 20 is interstitially integrated within fibers 52 of the skin contact layer 11 in the designated portion 21. FIG. 1c illustrates an embodiment wherein the active ingredient segment 20 is provided as an active ingredient coating 61 layers 61a-n applied over the portion 21 of the skin-contact surface 11, shown generally at 60. In this manner, the integration may be achieved during the manufacturing process by various methods, including by roll or spray application onto the skin contact layer 11 during production of the diaper 10, in a manner so as to provide layers 61a, 61b of the active ingredient. Particles of the active ingredient thus result in a series of layers 61a-n that yield a relatively tacky active ingredient segment 20. Preferably, there is at least one coating layer 61a, more preferably there is at least three coating layers 61a-c or a plurality of coating layers 61a-n. The thickness of each individual layer 61a, 61b may vary from one another, or may be equal to one another. Preferably, each individual layer 61a-61n has an individual thickness ranging between 0.025 cm up to 1.27 cm in thickness {each}, preferably ranging from 0.025 cm up to 0.635 cm {each}. Preferably, the overall thickness of the sum of the layers 61a-n combined should optimally not exceed 1.27 cm. In this embodiment, when diapering, optimal coverage can be gained by the caregiver swiping the active ingredient segment 20 against the infant's bottom and then closing the diaper. Thus, initial delivery is achieved when the diaper is being placed on the infant, and continuous delivery is achieved as the diaper is being worn and the active ingredient segment 20 rubs against or comes into contact with the infant's bottom.

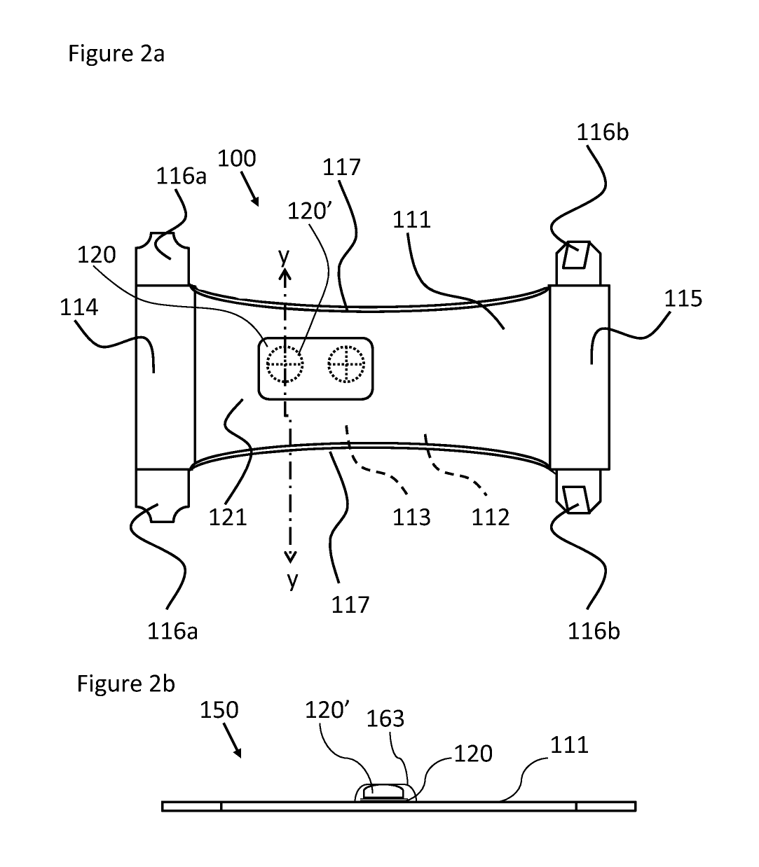

FIG. 2a illustrates a top view of an embodiment of the diaper having active ingredients integrated therein comprising a skin-contact surface appointed for contact with an infant's bottom, at least one inner absorption layer, an outer most layer, and an active ingredient segment, wherein the active ingredient segment is an active ingredient coating applied on the top of the portion of the skin-contact surface (as shown in FIG. 1c) and a removable peel tab is located thereon, shown generally at 100. FIG. 2b illustrates an embodiment, via a side cross-sectional view of the diaper having active integrated therein of FIG. 2a taken along line y-y, showing the active ingredient segment coating applied on the top of the portion of the skin-contact surface and the removable peel tab in place, appointed to be removed when the diaper is being donned, shown generally at 150. Diaper 100 comprises a skin-contact surface 111 appointed for contact with an infant's bottom, at least one inner absorption layer 112, and an outer most layer 113. Diaper 100 is constructed having a front waist band 114 and a back waist band 115 with mating attachment means 116a, 116b, and leg perimeter zones 117. Diaper 100 further comprises an active ingredient segment 120′ located on a barrier film or wall 120. Barrier film or wall 120 in turn is located over a portion 121 of skin-contact surface 111, herein portion 121. The active ingredient segment 120′ includes active ingredients associated therewith and is adapted for contact and direct delivery to the infant's bottom when the diaper 100 is placed on and worn by the infant. The active ingredient segment 120′ is located in association with the skin-contact surface 111 of the diaper 100 so that the active ingredients are applied as a coating preferably composed of a plurality of layers. Particles of the active ingredient thus result in a series of layers (a+n) that yield a relatively tacky active ingredient segment 120. Further provided is a covering 163 that covers and protects the active ingredients of segment 120′ from drying out or degrading during non-use or in storage. Removably peel tab 163 is generally comprised as a peel tab having a smooth, non-porous surface, or smooth surface with a polymeric coating or wax coating, or the like, that comes into intimate contact with the active ingredient segment 120 and can readily be peeled therefrom; conversely, the top of the peel tab 163 may be a non-coated surface. The peel tab 163 is similar to those utilized when dealing with tacky surfaces or adhesive surfaces. In this embodiment, when diapering, the caregiver simply opens the diaper 100 as usual and aligns the diaper 100 with the infant's bottom and peels back peel tab 163 and removes same to expose the active ingredient segment 120′. Optimal coverage can be gained by the caregiver then swiping the active ingredient segment 120 against the infant's bottom and then closing the diaper. Thus, initial delivery is achieved when the diaper is being placed on the infant, and continuous delivery is achieved as the diaper is being worn and the active ingredient segment 120′ rubs against or comes into contact with the infant's bottom.

FIG. 3 illustrates a view of an embodiment wherein the barrier wall 3020 and visa vie the active ingredient segment 3020′, is a raised surface located on said skin-contact surface of said diaper and is shaped as a decorative design.

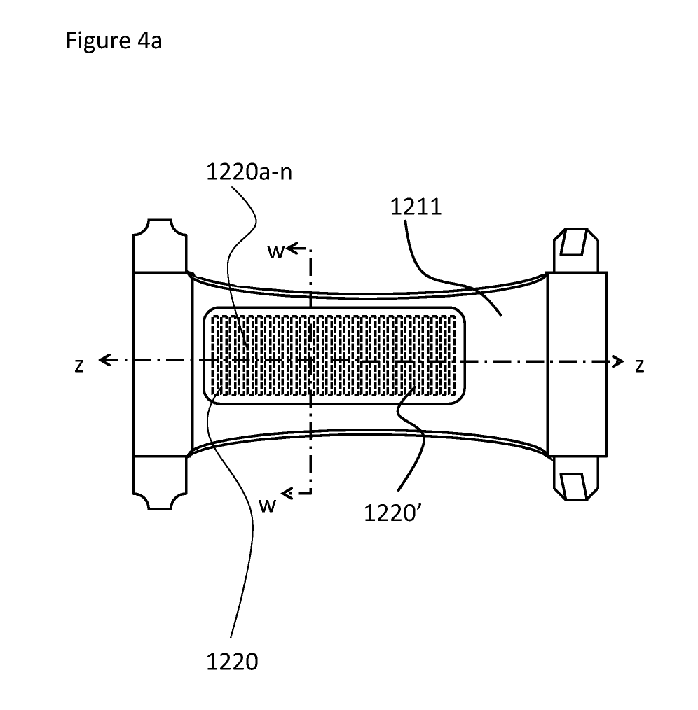

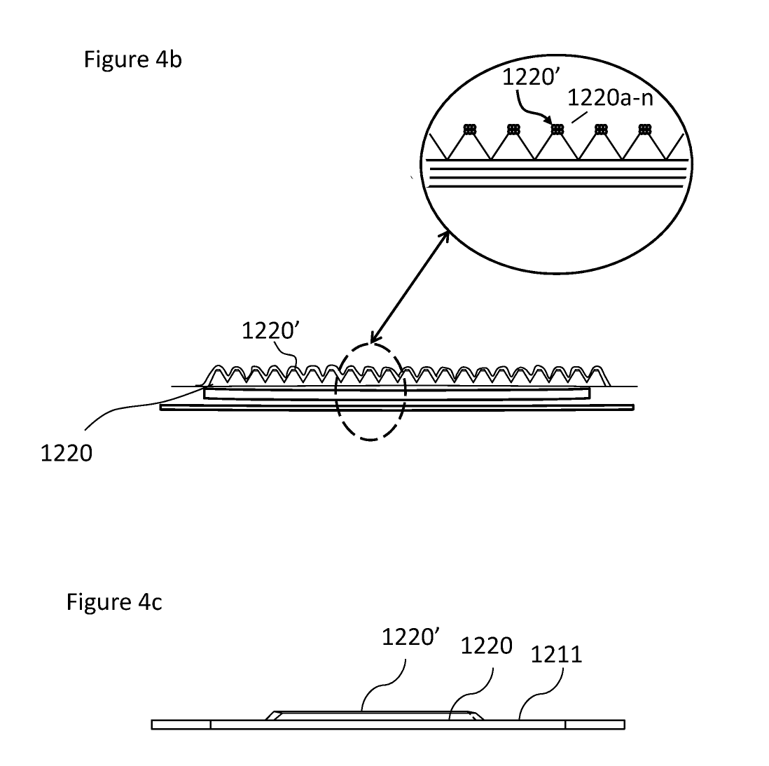

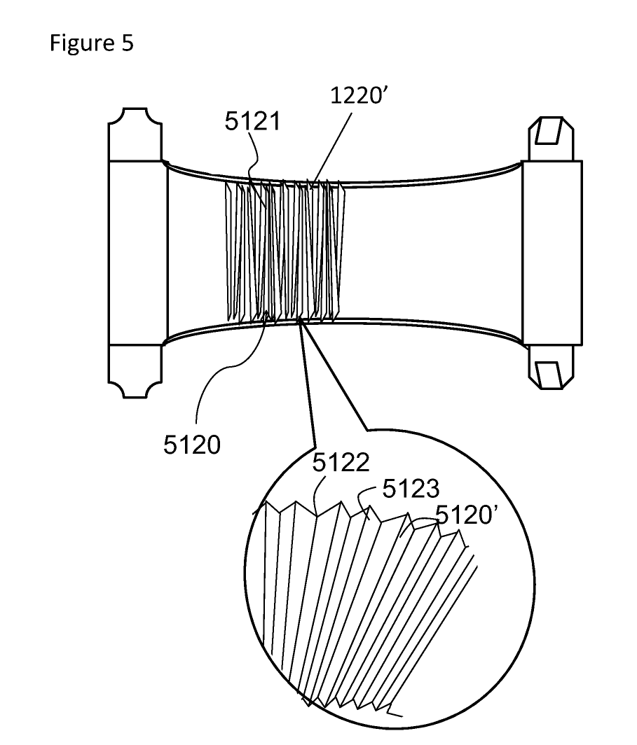

FIG. 4a illustrates a top view of an embodiment of the diaper having active ingredients integrated therein wherein the barrier wall consists essentially of a plurality of separate barrier wall segments that are closely dispersed but separate in relation to one another. The portion of the skin-contact surface 1211 includes a barrier wall 1220 having a plurality of separate barrier wall segment 1220a-n closely dispersed like comb bristles and having the active ingredient segment 1220′ located on the upper top surface of the segments 1220a-n. FIG. 4b illustrates a cross-sectional view taken along line z-z; FIG. 4c illustrates a cross-sectional view taken along line w-w.

FIG. 5 illustrates a top view of an embodiment of the diaper having active ingredients integrated therein wherein the barrier wall 5120 is formed as an accordion shaped portion 5121 having channels 5122 and peaks 5123 formed therein. Preferably, the active ingredient segment 5120′ is located substantially along the peaks 5123. In this manner, the active ingredient segment 5120′ does not cause any absorbency issues with the diaper because it is placed above and separate from the top diaper surface. In a preferably embodiment the active ingredient segment 5120′ is only located along the peaks 5123 and no active ingredients are located within the channels 5122. In a preferred embodiment the barrier wall 5120 and visa vie shaped portions 5121 are composed of a thin absorptive material enhancing absorbency abilities to provide extra absorbance ability to the diaper. A protective layer that dissolves and or a peal tab may be provided over the accordion portions. The accordion portions are preferably made of a resilient fiber material and/or can have a fibrous filling between each ridge. This actually will help with absorption (even more absorption) as it creates a channel effect to absorb urine but also absorb urine and/or fecal matter into channels wick the waste away from the skin and away from the infant's bottom. Preferably, a film layer is coated on the peaks and/or channel to wick urine and/or fecal matter into the channels.

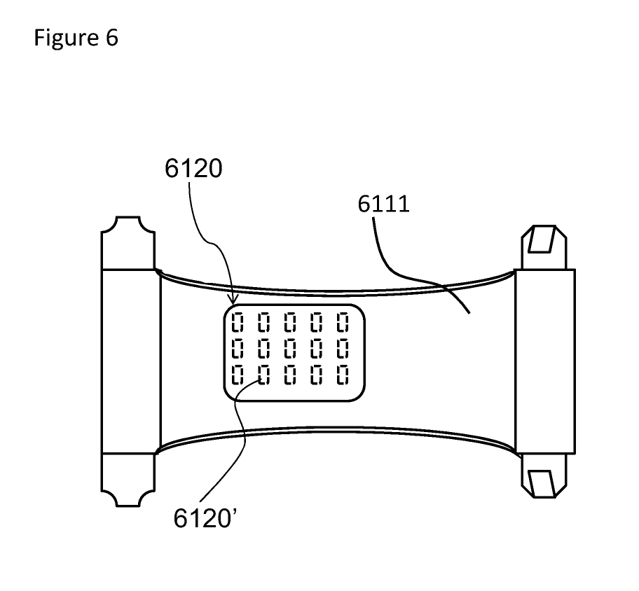

FIG. 6 illustrates a top view of an embodiment of the diaper having active ingredients integrated therein wherein the barrier wall 6120 is inset within the skin-contact surface 6111 of the diaper at a position below the skin-contact surface. The barrier wall 6120 is shown as a plurality of small insets each with active ingredients thereon forming the active ingredient segment 6120′.

In operation, the caregiver/parent removes the infant's soiled diaper and cleans the infant with diaper wipes or the like, or gives the infant a bath. After the infant is cleaned, using conventional methods, the caregiver would have to open the diaper ointment, or powder, etc. and topically apply the ointment or powder onto the infant, followed by diapering of the infant. However, in operation of the diaper with active ingredients herein, the caregiver simply opens the diaper to expose the skin-contact surface and slips the diaper under the infant, wherein a peel tab is provided over the active ingredients, the tab is removed, and the caregiver closes the front and back waist bands of the diaper around the infant's hips and fasteners same with fasteners.

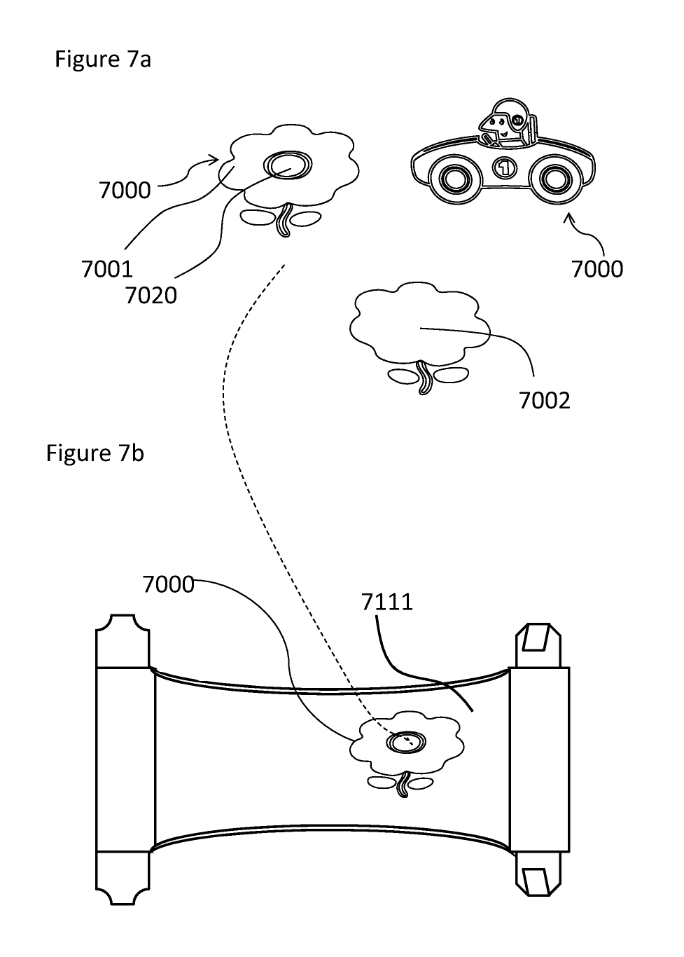

FIG. 7a illustrates a top view of an embodiment of the diaper active ingredient insert 7000 that has a top surface 7001 with a layer or coating of active ingredients 7020 therein. The bottom surface, shown at 7002, of the insert 7000 has an adhesive layer so that the insert 7000 can be readily stuck onto a diaper surface 7111 by a care giver to provide direct delivery of diaper rash ointment/or other treatments. The insert 7000 may be used by adults as well wherein the insert is placed within underwear. Active ingredients may include diaper rash ointment, hemorrhoid treatment cream, etc. FIG. 7b illustrates the diaper active ingredient insert adhered onto a diaper. The insert 7000 is preferably composed of a very thin paper or cotton material and is adapted to only be used once, and thrown away with the soiled diaper. The insert 7000 is appointed to come in an array of shapes, sizes, colors and designs.

Having thus described the invention in rather full detail, it will be understood that such detail need not be strictly adhered to, but that additional changes and modifications may suggest themselves to one skilled in the art, all falling within the scope of the invention as defined by the subjoined claims.

What is claimed is:

1. A diaper having active ingredients integrated therein, comprising:
a. a skin-contact surface appointed for contact with an infant's bottom, at least one inner absorption layer, and an outer most layer;
b. a barrier wall located on at least a portion of said skin-contact surface;
c. an active ingredient segment having said active ingredients integrated therein located on top of said barrier wall;
wherein said active ingredient segment is adapted to directly deliver said active ingredients to a skin surface when said diaper is placed on and worn.

2. A diaper having active ingredients integrated therein as recited by claim 1, wherein said barrier wall is a thin impervious layer preventing contact of said active ingredient segment and visa vie active ingredients with said skin-contact surface of said diaper.

3. A diaper having active ingredients integrated therein as recited by claim 1, wherein said barrier wall is a thin layer dissolvable when in contact with urine and/or body fluids.

4. A diaper having active ingredients integrated therein as recited by claim 1 comprising an exterior protective layer located over said active ingredient segment and containing said active ingredients integrated therein.

5. A diaper having active ingredients integrated therein as recited by claim 1, wherein said barrier wall consists essentially of a plurality of separate barrier wall segments that are closely dispersed but separate in relation to one another.

6. A diaper having active ingredients integrated therein as recited by claim 1, wherein said barrier wall is formed as an accordion shaped portion having channels and peaks formed therein.

7. A diaper having active ingredients integrated therein as recited by claim 6, wherein said active ingredient segment is located substantially along said peaks.

8. A diaper having active ingredients integrated therein as recited by claim 6, wherein said active ingredient segment is located only along said peaks and no active ingredients are located within said channels.

9. A diaper having active ingredients integrated therein as recited by claim 6, wherein said accordion shaped portion is composed of a thin absorptive material.

10. A diaper having active ingredients integrated therein as recited by claim 6 comprising an exterior protective layer.

11. A diaper having active ingredients integrated therein as recited by claim 1, wherein said active ingredients is a diaper rash ointment for treatment or prevention of diaper rash.

12. A diaper having active ingredients integrated therein as recited by claim 1, wherein said portion of said skin-contact surface associated with said active ingredient segment is only located on a soiling zone extending from a crotch area to a buttocks area of said diaper so that said active ingredient segment provides contact and dissemination of said active ingredients to crotch and buttocks areas of said infant while said diaper is being donned and worn.

13. A diaper having active ingredients integrated therein as recited by claim 1, wherein said active ingredient segment is applied as a coating upon said portion of said skin-contact surface to provide transfer of said active ingredients, said coating being comprised of at least one active ingredient layer.

14. A diaper having active ingredients integrated therein as recited by claim 13 comprising a removably peel tab located over said coating of said active ingredients to prevent degradation or drying of said active ingredients during storage and before use.

15. A diaper insert sheet having active ingredients integrated therein appointed to be inserted within a diaper, comprising:
a. a thin sheet having a top wall appointed for contact with an infant's skin and a bottom wall having an adhesive coating applied thereto being appointed to be adhered to a skin-contact surface of said diaper;
b. an active ingredient segment having said active ingredients integrated therein located on said top wall of said sheet;
wherein said active ingredient segment is adapted to directly deliver said active ingredients to a skin surface when said diaper is placed on and worn.

16. A diaper insert sheet having active ingredients integrated therein as recited by claim 15, wherein said active ingredients is a diaper rash ointment for treatment or prevention of diaper rash.

17. A diaper insert sheet having active ingredients integrated therein as recited by claim 15, wherein said active ingredient segment is applied as a coating upon said top layer to provide transfer of said active ingredients, said coating being comprised of at least one active ingredient layer.

18. A sheet having active ingredients integrated therein, consisting essentially of:
a. a top sheet surface appointed to be wiped against and come into direct contact with an infant's skin and a bottom sheet surface; and
b. an active ingredient segment having said active ingredients integrated therein located on at least a portion of said top sheet surface;
wherein said active ingredient segment is adapted to directly delivery said active ingredients to said infant's skin when said top sheet surface is wiped against said infant in preparation for diapering said infant.

19. A sheet having a active ingredients integrated therein as recited by claim 18, wherein said active ingredients is a diaper rash ointment for treatment or prevention of diaper rash, wherein said diaper rash ointment is a coating located on said top sheet surface.

20. A sheet having active ingredients integrated therein as recited by claim 18, wherein said active ingredients is selected form a group consisting of baby oil, petroleum jelly or lotion for treatment or prevention of dry skin.
